(19) United States
(12) Patent Application Publication
(10) Pub. No.: US 2024/0013307 A1
(43) Pub. Date: Jan. 11, 2024

(54) SYSTEMS AND METHODS FOR FRAUDULENT CLAIM DETECTION

(71) Applicant: Change Healthcare Holdings, LLC, Nashville, TN (US)

(72) Inventor: Subba Reddy Appala, Mt. Juliet, TN (US)

(21) Appl. No.: 17/860,378

(22) Filed: Jul. 8, 2022

(51) Int. Cl. G06Q 40/08; G06Q 20/02

(52) U.S. Cl. CPC G06Q 40/08; G06Q 20/023

(57) ABSTRACT

In an embodiment, systems and methods for identifying fraudulent claims is provided. When a claim is received from a medical provider, contact information associated with a patient is determined. The contact information is used to generate and send a message to the patient asking the patient to confirm that the claim is not fraudulent or asking the patient to confirm certain details associated with the claim such as the associated medical procedures, the date of the procedures, and the names of any medical providers associated with the medical procedures. If the patient confirms the claim, the claim may be processed normally using an auto-adjudication process. If the patient cannot confirm the claim or certain aspects of the claim, the claim may be flagged for further review and fraud processing or may even be denied.

### BACKGROUND

In the United States, approximately 700 billion dollars is lost to fraudulent healthcare claims annually. While there exists solutions that can review claims for potentially fraudulent claims, such software is unable to identify all fraudulent claims. Moreover, often once fraudulent claims are identified, the process to recover payments made on those claims is costly and time consuming. Accordingly, there is a need for a claims processing system that can quickly and accurately identify fraudulent claims before they are paid.

### SUMMARY

In an embodiment, systems and methods for identifying fraudulent claims are provided. According to one embodiment, when a claim is received from a medical provider for services provided to a patient, contact information associated with the patient is determined. The contact information is used to generate and send a message to the patient asking the patient to confirm that the claim is not fraudulent or that certain details associated with the claim, such as the associated medical procedures, the date of the procedures, and the names of any medical providers associated with the medical procedures, are accurate. If the patient confirms the claim, the claim may be processed normally using an auto-adjudication process. If the patient cannot confirm the claim or certain aspects of the claim, the claim may be flagged for further review and fraud processing, or even denied.

The systems and methods described herein provide at least the following advantages. First, because the patient is asked to review claims as they are received, potentially fraudulent claims can be identified early in the claims processing pipeline. Second, because the patient is directly involved in the claims review process, the mislabeling of non-fraudulent claims as fraudulent is reduced.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate a fraudulent claims detection system and method. Together with the description, the figures further serve to explain the principles of the fraudulent claims detection system and method described herein and thereby enable a person skilled in the pertinent art to make and use the fraudulent claims detection system and method.

FIG. 1 is an example environment for detecting fraudulent claims;

FIG. 2 is an illustration of a method for detecting fraudulent claims;

FIG. 3 is an illustration of another example method for detecting fraudulent claims; and

FIG. 4 shows an example computing environment in which example embodiments and aspects may be implemented.

### DETAILED DESCRIPTION

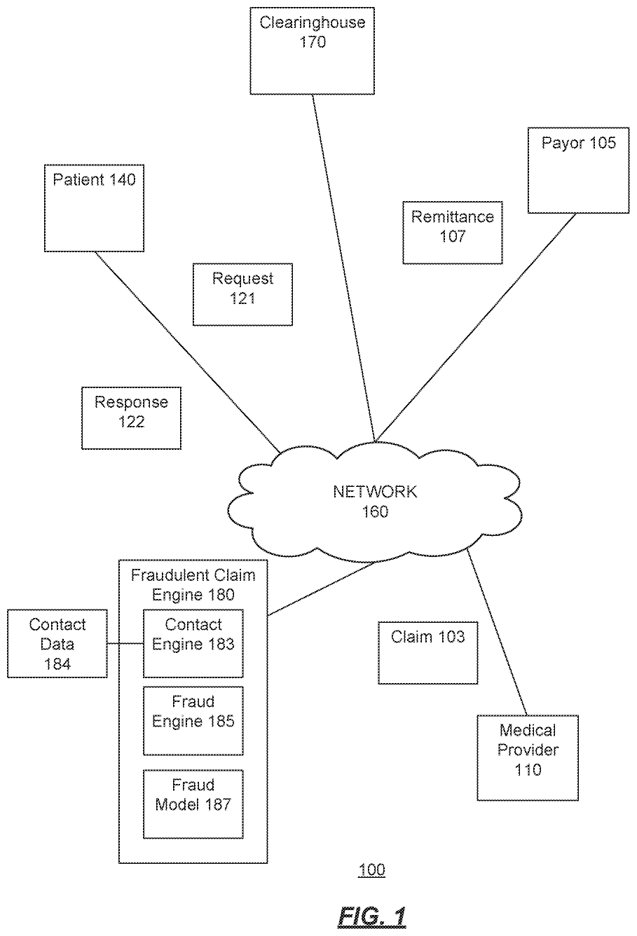

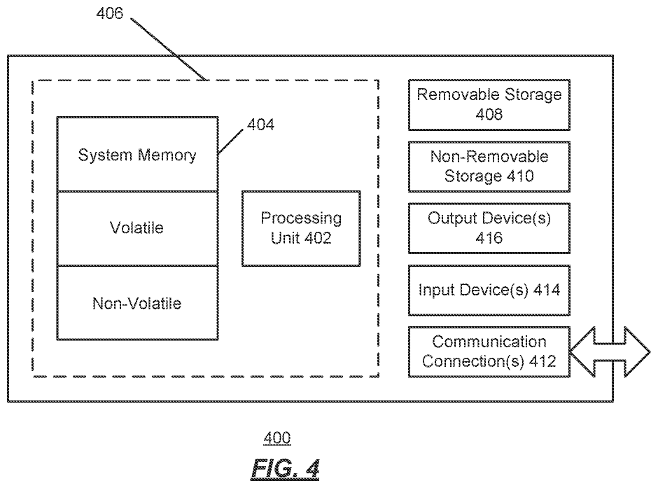

FIG. 1 is an example environment 100 for detecting fraudulent claims. As shown, the environment 100 may include a clearinghouse 170, one or more medical providers 110, one or more payors 105 and one or more patients 140 in communication through a network 160. The network 160 may include a combination of private networks (e.g., LANs) and public networks (e.g., the Internet). Each of the clearinghouse 170, the medical provider 110, the patient 140, and the payor 105 may use, or may be partially implemented by, one or more general purpose computing devices such as the computing device 400 illustrated in FIG. 4

The clearinghouse 170 may be a medical claims clearinghouse 170 and may receive claims 103 for medical services rendered by medical providers 110 to patients 140. The clearinghouse 170 may then submit each received claim 103 to a corresponding payor 105 (e.g., insurance company or government entity), and may receive remittances 107, or claim payment decisions (e.g., denied, accepted, accepted at some level) from the payors 105 for the claims 103. The clearinghouse 170 may further facilitate transfer of the remittances 107 to the medical providers 110.

As described above, one drawback associated with current health systems is fraudulent claims 103. Fraudulent claims 103 submitted by medical providers 110 may take a variety of forms. One type of fraudulent claim 103 is for medical services that were never provided. For example, a patient 140 may visit a medical provider 110 for a checkup. The medical provider 110 may submit a claim 103 for a medical service such as an X-ray that was not performed during the visit.

Another type of fraudulent claim 103 is the use of non-doctors to provide certain medical services yet submitting a claim 103 for the cost of the services as if they were provided by a doctor. Continuing the example above, a nurse may have performed some or all of the checkup for the patient 140, yet the medical provider 110 may have submitted a claim 103 for the checkup as if it was performed by a doctor.

Another type of fraudulent claim is known as up-coding or up-billing where a claim 103 is made for a more costly medical service than was provided. For example, a patient 140 may have received an X-Ray from a medical provider 110, yet the medical provider 110 may have submitted a claim 103 for a more costly MRI procedure.

Accordingly, to solve this problem, the environment 100 may further include a fraudulent claim engine 180 that identifies possible fraudulent claims 103 for further review. As shown, the fraudulent claim engine 180 includes serval components including, but not limited to, a contact engine 183, a fraud engine 185, and fraud model 187. More or fewer component may be supported. Each component of the fraudulent claim engine 180 may be implemented together or separately using one or more general purpose computing devices such as the computing device 400 illustrated with respect to FIG. 4. Note that while the fraudulent claim engine 180 is shown as being separate from the clearinghouse 170 it is for illustrative purposes only; in some embodiments the fraudulent claim engine 180 may be part of the clearinghouse 170.

The contact engine 183 may collect contact data 184 for one or more patients 140. The contact data 184 for a patient 140 may include information that can be used by the fraudulent claim engine 180 to contact the patient 140. This information may include the phone number, email address, and mailing address of the patient 140. The information may further include social media account names or handles that may be used to contact the patient 140. Other information may be supported.

In some embodiments, the contact engine 183 may collect the contact data 184 for patients 140 by first identifying each patient 140 based on the claims 103 received by the clearinghouse 170. The contact engine 183 may then use patient enrolment information provided by one or more payors 105 for each patient 140 to determine the contact data 184 for each identified patient 140. Each payor 105 may provide enrolment information for their patients 140 that includes information about each patient 140 including their address and other contact data 184.

Alternatively, or additionally, some or all of the patients 140 may provide their contact data 184 to the contact engine 183. For example, patients 140 may create an account with fraudulent claim engine 180 to receive information about their claims 103 and may provide their contact data 184 as part of the account creation process. Where a patient 140 is a minor or other dependent, a parent or caregiver may provide their own contact data 184 for purposes of account creation.

The fraud engine 185 may receive an indication of a claim 103 submitted for a patient 140, and in response to the indication, the fraud engine 185 may perform a process to determine if the claim 103 is fraudulent. In some embodiments, when a claim 103 is received by the clearinghouse 170 from a medical provider 110, an indication of the claim 103 (or the claim 103 itself) is provided by the clearinghouse 170 to the fraud engine 185.

In some embodiments, upon receiving the claim 103, the fraud engine 185 may automatically generate a request 121 and may provide the request 121 to the patient 140 associated with the claim 103. The request 121 may be a request for the patient 140 to confirm or deny the claim 103. In some embodiments, the fraud engine 185 may extract details from the claim 103 to be included or presented in the request 121 using plain non-technical language. The request 121 may include information such as the medical procedure associated with the claim 103, the date and location associated with the claim 103, and the name of any doctors or medical professionals associated with the claim 103.

For example, the fraud engine 185 may receive a claim 103 for a chest X-ray performed for the patient 140 by a Dr. Smith, on Nov. 28, 2021, in Albuquerque New Mexico, at First General Hospital. In response, the fraud engine 185 may generate a request 121 that includes text such as “Did you receive a chest X-ray from Dr. Smith on Nov. 28, 2021, at First General Hospital in Albuquerque New Mexico?” The request 121 may further include user interface elements labeled “Yes” and “No” that the user may select to either confirm or deny the claim 103. Depending on the embodiment, the request 121 may further include a text box through which the patient 140 may provide additional or clarifying information about the claim 103. For example, the patient 140 may enter text clarifying that the medical procedure was performed at a different date or by a different medical professional.

In some embodiments, rather than ask the user/patient to confirm or deny the entire claim 103, the request 121 may ask the patient 140 to confirm or deny multiple aspects of the claim 103. Continuing the example above, the request 121 may include multiple questions such as “Did you receive a medical service on Nov. 28, 2021 at First General Hospital in Albuquerque New Mexico?”, “Was the medical service a chest X-Ray?”, and “Was the X-ray read by Dr. Smith?” Each question may include user interface elements labeled “Yes” and “No” that the patient 140 may select to either confirm or deny the associated aspect of the claim 103.

In some embodiments, the request 121 may be provided to the patient 140 at an address and/or communication channel indicated by the contact data 184. In other embodiments, the request 121 may be provided in an app or specialized application that the patient 140 may access using their computing device or smartphone. The app or application may be provided by the fraudulent claim engine 180. Alternatively, the app or application may be provided by a payor 105 (e.g., insurance company) who provides medical coverage to the patient 140.

In some embodiments, the fraud engine 185 may automatically send a request 121 for every claim 103 received for each patient 140. Alternatively, the fraud engine 185 may send requests 121 only for those claims 103 that show some other signs of being fraudulent. For example, certain medical procedures or medical providers 110 may have a history of being associated with fraudulent claims 103. As another example, when a claim 103 indicates that the medical procedure was performed at a location that is far from where the patient 140 lives, the claim 103 may be more likely to be fraudulent. Depending on the embodiment, the fraud engine 185 may assign a potential fraud score to each claim 103 and may generate a request 121 for those claims 103 with a potential fraud score that exceeds a threshold.

In some embodiments, the claim 103 may receive a potential fraud score from a fraud model 187. The fraud model 187 may be a machine learning model trained to identify possible fraudulent claims 103. Any method for constructing a machine learning model may be used.

After receiving the request 121, the patient 140 may generate a response 122. The response 122 may either indicate that the entire claim 103 is correct or incorrect or may indicate that certain aspects or facts of the claim 103 are correct or incorrect. Depending on the embodiment, when a response 122 is received that confirms the claim 103, the fraud engine 185 may send a message to the clearinghouse 170, and the claim 103 may sent for auto-adjudication. Auto-adjudication is an automated process through which a claim 103 is processed and fulfilled.

If the response 122 indicates that the claim 103 is not confirmed (i.e., is incorrect) by the patient 140 (in whole or in part) the fraud engine 185 may take several possible actions. In some embodiments, the fraud engine 185 or clearinghouse 170 may deny the claim 140. In other embodiments, the claim 103 and the response 122 may be sent to a human reviewer or investigator who may take further actions to determine whether or not the claim 103 is fraudulent. For example, the claim 103 may be provided to the payor 105 with a flag or other indication that the claim 103 may be fraudulent. The payor 105 may then undertake their own investigation of the claim 103.

In embodiments where a fraud model 187 was used to identify the claim as possibly being a fraudulent claim 103, the response 122 may be used as feedback or additional training data to update the model 187. For example, if the response 122 indicates that the claim 103 was not confirmed by the patient 140, the claim 103 and response 122 may be used as positive feedback for the model 187. Conversely, if the response 122 indicates that the claim 103 was confirmed by the patient 140, the claim 103 and response 122 may be used as negative feedback for the model 187.

As may be appreciated, because patients 140 may be busy or unwilling to confirm or deny requests 121, in some embodiments one or more actions may be taken when no response 122 is received from the patient 140. These actions may include assuming the claim 103 is likely non-fraudulent and sending the claim 103 for auto-adjudication, or assuming the claim 103 is likely fraudulent and sending the claim 103 for further review. The threshold duration of time that the fraud engine 185 may wait for a response 122 from a patient 140 may be set by a user or administrator. Example thresholds include one day, two days, one week, etc.

As may be appreciated, the fact that a patient 140 confirms a claim 103 does not necessarily prove that the claim 103 is not fraudulent. For example, a patient 140 who receives frequent medical care and has poor memory may assume that a claim 103 is valid and may confirm a claim 103 for care that they did not receive. Conversely, that a patient 140 denies a claim 103 does not necessarily indicate that a claim 103 is fraudulent. For example, a patient may forget the name of the doctor that performed a medical procedure and as a result may decline to confirm the claim 103.

Accordingly, in some embodiments, rather than have the response 122 be dispositive of whether a claim 103 is fraudulent, a fraud model 187 is used to determine if the claim 103 is likely fraudulent. The model 187 may receive as an input the response 122 (if any) from the patient 140, and other information about the claim 103, and may output a score or probability that the claim 103 is fraudulent. Claims 103 with scores that are below a threshold score may be sent to auto-adjudication, while claims 103 with scores that are above the threshold may be denied or may be sent for further review as described above. The fraud model 187 may be the same or different model 187 than was used to determine if a claim 103 should be confirmed by the patient 140.

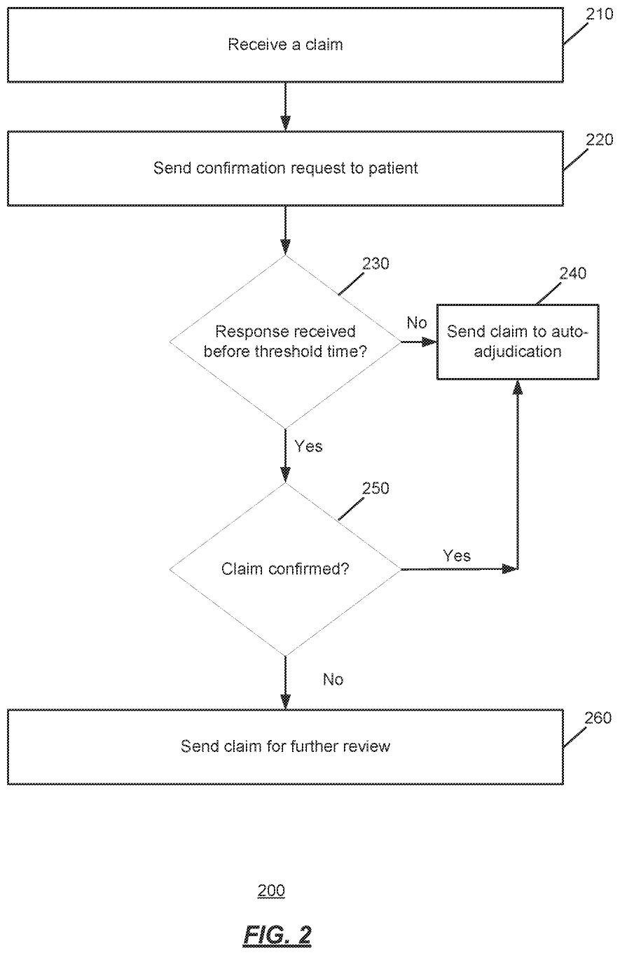

FIG. 2 is an illustration of an example method 200 for detecting fraudulent claims. The method 200 may be implemented by the fraudulent claim engine 180.

At 210, a claim is received. The claim 103 may be received by the clearinghouse 170 from a medical provider 110. The claim 103 may be associated with a patient 140 and may identify a medical service or procedure performed by a medical provider for the patient 140.

At 220, a confirmation request is sent to the patient. The confirmation request 121 may be sent by the fraud engine 185 to the patient 140. The request 121 may be an electronic document (e.g., e-mail, SMS, and notification from a replated app or application) and may request that the patient 140 confirm the claim 103 or certain details about the claim 103. For example, the patient 140 may be asked to confirm the date associated with the claim 103, a location associated with the claim 103, the medical procedure or service associated with the claim 103, and the doctor or physician that performed the associated medical service or procedure. The confirmation request 121 may be sent automatically, or only after the claim 103 has been identified or flagged as potentially fraudulent (as discussed in more detail above and below).

At 230, whether a response to the request has been received is determined. The determination may be made by the fraud engine 185. In some embodiments, if no response has been received and a threshold amount of time has passed, then the method 200 may continue at 240. Else the method 200 may continue at 250. The threshold time passing may indicate that the patient 140 is either unwilling or unable to confirm or deny the associated medical claim 103.

At 240, the claim is sent to auto-adjudication. The claim may be sent to auto-adjudication by the fraud engine 185. In some embodiments, the claims may be sent to auto-adjudication when no response has been received from the patient 140 only where the claim was not flagged as potentially fraudulent (as discussed below) or when the fraud model 187 has otherwise indicated that the claim 103 is not fraudulent or has a fraud score that is below a threshold.

If a response has been received before the threshold of time has passed, at 250, a determination of whether the claim was confirmed by the patient 140 is made. The determination may be made by the fraud engine 185. The fraud engine 185 may determine if the claim 103 was confirmed by processing the response 122 received from the patient 140. If the claim 103 is confirmed by the fraud engine 185, the method 200 may continue at 240 where the claim 103 may be sent for auto-adjudication.

If the fraud engine 185 determines that the claim was not confirmed (in whole or in part) based on the patient's response 122, at 260, the claim is sent to the payor 105 for further review. The claim may be sent for further review by the fraud engine 185. Because the claim 103 was at least partially denied or not confirmed by the patient 140, the claim 103 may receive a manual review for fraud by the payor 105. Alternatively, the claim 103 may be denied or returned to the medical provider 110 that submitted the claim 103.

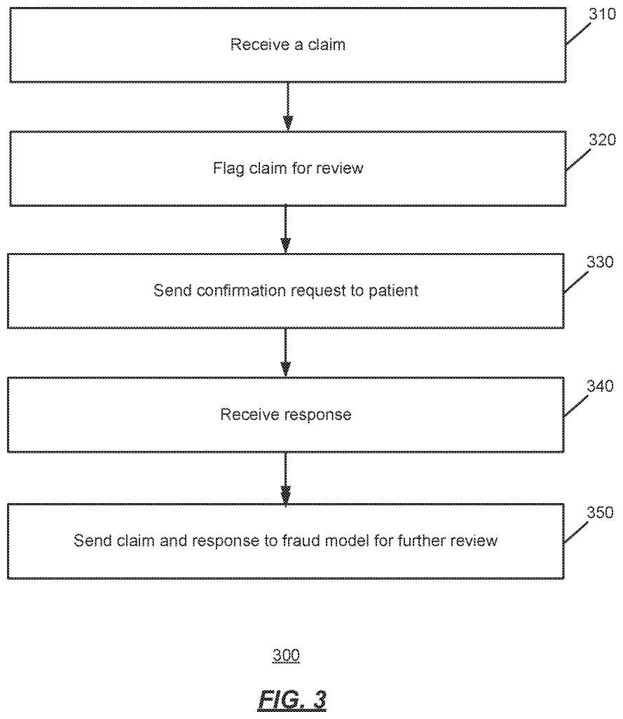

FIG. 3 is an illustration of another example method 300 for detecting fraudulent claims. The method 300 may be implemented by the fraudulent claim engine 180.

At 310, a claim is received. The claim 103 may be received by the clearinghouse 170 from a medical provider 110. The claim 103 may be associated with a patient 140 and may identify a medical service or procedure performed by a medical provider for the patient 140.

At 320, the claim is flagged for review. The claim 103 may be flagged for further review by the fraud engine 185. In some embodiment, every claim 103 may be flagged for review by the associated patient 140. In other embodiments, claims 103 having certain characteristics or certain criteria may be flagged for review. For example, certain associated medical procedures or medical providers 110 may be associated with fraud and may cause a claim 103 to be flagged for review. In some embodiments, a fraud model 187 may be used to flag a claim 103 for review.

At 330, a confirmation request is sent to the patient. The confirmation request 121 may be sent by the fraud engine 185 to the patient 140 by the fraud engine 185. The request 121 may be an electronic document (e.g., e-mail, SMS, and notification from a replated app or application) and may request that the patient 140 confirm the claim 103 or certain details about the claim 103. For example, the patient 140 may be asked to confirm the date associated with the claim 103, a location associated with the claim 103, the medical procedure or service associated with the claim 103, and the doctor or physician that performed the associated medical service or procedure.

At 340, a response is received. The response 122 may be received by the fraud engine 185. The response 122 may confirm or deny the claim 103 or certain aspects of the claim 103. The response 122 may be generated in response to the patient 140 selecting or activating one or more user-interface elements in the request 121.

At 350, the claim and response are sent to a fraud model for further review. The fraud model 187 may receive as an input the claim 103 and the response 122, and may output a potential fraud score. Depending on the potential fraud score, the fraud engine 185 may determine whether to send the claim 103 for auto-adjudication, or to send the claim 103 for further review

FIG. 4 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 4, an example system for implementing aspects described herein includes a computing device, such as computing device 400. In its most basic configuration, computing device 400 typically includes at least one processing unit 402 and memory 404. Depending on the exact configuration and type of computing device, memory 404 may be volatile (such as random access memory (RAM)), non-volatile (such as read-only memory (ROM), flash memory, etc.), or some combination of the two. This most basic configuration is illustrated in FIG. 4 by dashed line 406.

Computing device 400 may have additional features/functionality. For example, computing device 400 may include additional storage (removable and/or non-removable) including, but not limited to, magnetic or optical disks or tape. Such additional storage is illustrated in FIG. 4 by removable storage 408 and non-removable storage 410.

Computing device 400 typically includes a variety of computer readable media. Computer readable media can be any available media that can be accessed by the device 400 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 400. Any such computer storage media may be part of computing device 400.

Computing device 400 may contain communication connection(s) 412 that allow the device to communicate with other devices. Computing device 400 may also have input device(s) 414 such as a keyboard, mouse, pen, voice input device, touch input device, etc. Output device(s) 416 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
receiving a claim by a computing device, wherein the claim is associated with a patient;
determining contact information associated with the patient by the computing device;
generating a confirmation request for the claim by the computing device;
sending the confirmation request to the patient based on the contact information by the computing device;
receiving a confirmation that the claim is correct from the patient by the computing device; and
in response to receiving the confirmation, sending the claim to auto-adjudication by the computing device.

2. The method of claim 1, further comprising receiving information indicating that the claim is incorrect from the patient by the computing device; and
in response to receiving the information indicating that the claim is incorrect, sending the claim for review.

3. The method of claim 1, further comprising receiving information indicating that the claim is incorrect from the patient by the computing device; and
in response to receiving the information indicating that the claim is incorrect, rejecting the claim.

4. The method of claim 1, further comprising:
determining that a threshold amount of time has passed since the confirmation request was sent to the patient and no response to the request was received;
determining that the claim is likely not fraudulent; and
in response to the determination that the claim is likely not fraudulent, sending the claim to auto-adjudication.

5. The method of claim 1, further comprising:
determining that the claim may be a fraudulent claim, wherein generating the confirmation request for the claim is in response to the determination that the claim may be a fraudulent claim.

6. The method of claim 5, wherein the claim is associated with a medical service and wherein determining that the claim may be a fraudulent claim further comprises determining that the claim may be a fraudulent claim based on the medical service.

7. The method of claim 5, wherein the patient is associated with a location, and wherein determining that the claim may be a fraudulent claim further comprises determining that the claim may be fraudulent based on the location of the patient and a location associated with a medical service associated with the claim.

8. The method of claim 1, further comprising automatically sending the confirmation request to the patient in response to receiving the claim.

9. The method of claim 1, wherein the claim is a medical claim, and the computing device is associated with a medical claim clearinghouse.

10. A system comprising:
at least one computing device; and
a computer-readable medium with computer-executable instructions stored thereon that when executed by the at least one computing device cause the system to:
receive a claim, wherein the claim is associated with a patient;
determine contact information associated with the patient;
generate a confirmation request for the claim;
send the confirmation request to the patient based on the contact information;
receive a confirmation that the claim is correct from the patient; and
in response to receiving the confirmation, send the claim to auto-adjudication.

11. The system of claim 10, further comprising computer-executable instructions stored thereon that when executed by the at least one computing device cause the system to:
receive information indicating that the claim is incorrect from the patient; and
in response to receiving the information indicating that the claim is incorrect, send the claim for review.

12. The system of claim 10, further comprising computer-executable instructions stored thereon that when executed by the at least one computing device cause the system to:
receive information indicating that the claim is incorrect from the patient by the computing device; and
in response to receiving the information indicating that the claim is incorrect, reject the claim.

13. The system of claim 10, further comprising computer-executable instructions stored thereon that when executed by the at least one computing device cause the system to:
determine that a threshold amount of time has passed since the confirmation request was sent to the patient and no response to the request was received;
determine that the claim is likely not fraudulent; and
in response to the determination that the claim is likely not fraudulent, send the claim to auto-adjudication.

14. The system of claim 10, further comprising computer-executable instructions stored thereon that when executed by the at least one computing device cause the system to:
determine that the claim may be a fraudulent claim, and wherein the computer-executable instructions that cause the system to generate a confirmation request for the claim further cause the system to generate the confirmation request in response to the determination that the claim may be a fraudulent claim.

15. The system of claim 14, wherein the claim is associated with a medical service, and wherein the computer-executable instructions stored thereon that when executed by the at least one computing device cause the system to determine that the claim may be a fraudulent claim are configured to determine that the claim may be a fraudulent claim based on the medical service.

16. The system of claim 14, wherein the patient is associated with a location, and wherein the computer-executable instructions stored thereon that when executed by the at least one computing device cause the system to determine that the claim may be a fraudulent claim are configured to determine that the claim may be a fraudulent claim based on the location of the patient and a location associated with a medical service associated with the claim.

17. The system of claim 10, further comprising computer-executable instructions stored thereon that when executed by the at least one computing device cause the system to: automatically send the confirmation request to the patient in response to receiving the claim.

18. The system of claim 10, wherein the claim is a medical claim, and the computing device is associated with a medical claim clearinghouse.

19. A non-transitory computer-readable medium with computer-executable instructions stored thereon that when executed by at least one computing device cause the at least one computing device to:
receive a claim, wherein the claim is associated with a patient;
determine contact information associated with the patient;
generate a confirmation request for the claim;
send the confirmation request to the patient based on the contact information;
receive a confirmation that the claim is correct from the patient; and
in response to receiving the confirmation, send the claim to auto-adjudication.

20. The computer-readable medium of claim 19, further comprising computer-executable instructions stored thereon that when executed by the at least one computing device cause the at least one computing device to:
receive information indicating that the claim is incorrect from the patient; and
in response to receiving the information indicating that the claim is incorrect, send the claim for review.
